(19) United States
(12) Patent Application Publication
(10) Pub. No.: US 2016/0287162 A1
(43) Pub. Date: Oct. 6, 2016

(54) METHOD AND APPARATUS FOR PROCESSING EEG SIGNALS

(71) Applicant: NEUROCHIP CORPORATION, Toronto, Ontario (CA)

(72) Inventors: Berj L. Bardakjian, Toronto (CA); Marija Cotic, Toronto (CA); Mirna Guirgis, Toronto (CA)

(73) Assignee: Neurochip Corporation, Toronto, Ontario (CA)

(21) Appl. No.: 15/034,441

(22) PCT Filed: Nov. 5, 2014
(86) PCT No.: PCT/CA14/00789

§ 371 Date: May 4, 2016

Related U.S. Application Data
(60) Provisional application No. 61/900,238, filed on Nov. 5, 2013.

(51) Int. Cl. A61B 5/00; A61B 5/0478

(52) U.S. Cl. CPC A61B 5/4094; A61B 5/0478; A61B 5/726; A61B 5/7475; A61B 5/6868

(57) ABSTRACT

A method of processing EEG signals obtained from a subject using a grid of electrodes comprises processing the EEG signals to determine a phase coherence measure of the EEG signals for selected electrode pairs of said grid; determining electrode pairs where strong phase coherence of the EEG signals is observed in a low frequency range during interictal activity; and using the determined electrode pairs to identify a possible seizure zone of the subject's brain.

### CROSS-REFERENCE TO RELATED APPLICATION

The subject application claims the benefit of U.S. Provisional Application No. 61/900,238 to Bardakjian et al. filed on Nov. 5, 2013, the entire content of which is incorporated herein by reference.

### FIELD

The subject application relates generally to a method and apparatus for processing EEG signals.

### BACKGROUND

Neural electrical oscillations have been observed in multiple studies in both humans and animals. These rhythms arise from the synchronous oscillations of large ensembles of neurons and are considered to play an important role in the integration of cortical processes. Several studies have shown a correlation between the oscillatory activity of the brain with such cognitive processes as memory, attention and consciousness [1]. In the pathological brain, neural rhythms similarly remain a strong focus as abnormal oscillatory patterns have been observed for several disorders including epilepsy, Parkinson's disease and schizophrenia [1, 2].

Electrical rhythms are being closely examined in relation to epilepsy as the pathological locking of seizure discharges indicates a disturbance in the normal functioning of neuronal oscillations. Several prominent brain rhythms have shown subtle physiological changes in the face of pathology, such as deceleration in their activities or malfunctions in their mechanisms of synchronization. During seizure episodes, 60 to 80 Hz activity has been shown to appear specific to epileptic network function, while ictal patterns contain noticeable activity in the beta and gamma ranges at seizure onset [2].

New advances in recording techniques have made it possible for large numbers of recording electrodes to be placed in the brain and sampled at very high rates (eg. >1 kHz). As a result, multi-contact and grid electrodes can be placed in distributed and concentrated areas of the human brain to record a larger ‘representation’ of the underlying neuronal electrical activity. The analysis of multi-site and high-sampled data can provide insights into the associations of various information processing areas and cellular layers. Furthermore, high sampling rates provide the ability to investigate high frequency activities (i.e. >80 Hz) throughout the brain.

Recently, high frequency oscillations (HFOs >80 Hz) have been reported in the human epileptic brain [3]. HFOs have been identified in neuronal tissues generating seizures, in patients with focal epilepsies [3, 4]. Their recent discovery has highlighted their potential involvement in pathological activity, necessitating a need for further analysis into their mechanisms of generation.

It is therefore an object to provide a novel method and apparatus for processing EEG signals.

### SUMMARY

Accordingly, in one aspect there is provided a method of processing EEG signals obtained from a subject using a grid of electrodes comprising processing the EEG signals to determine a phase coherence measure of the EEG signals for selected electrode pairs of said grid; determining electrode pairs where strong phase coherence of the EEG signals is observed in a low frequency range during interictal activity; and using the determined electrode pairs to identify a possible seizure zone of the subject's brain.

In some embodiments, the selected electrode pairs comprise all possible electrode pairs of the grid.

In some embodiments, during the processing, wavelet phase coherence of the EEG signals for the selected electrode pairs is calculated. The wavelet phase coherence may be calculated for frequencies between 1 Hz and 400 Hz at a resolution of about 1 Hz and may be calculated using a Morlet wavelet.

In some embodiments, the low frequency range comprises 8 Hz to 11 Hz. The method may further comprise, for the determined electrode pairs, determining if an increased phase coherence is observed in a high frequency range during seizure activity to verify the identification of the possible seizure zone. In this case, the high frequency range may comprise frequencies greater than 80 Hz.

According to another aspect there is provided a non-transitory computer-readable medium comprising program code for, when executed, processing EEG signals obtained from a subject using a grid of electrodes comprising program code for processing the EEG signals to determine a phase coherence measure of the EEG signals for selected electrode pairs of said grid; program code for determining electrode pairs where strong phase coherence of the EEG signals is observed in a low frequency range during interictal activity; and program code for using the determined electrode pairs to identify a possible seizure zone of the subject's brain.

According to yet another aspect there is provided an apparatus comprising memory storing executable instructions; and at least one processor communicating with the memory and executing the instructions therein to cause the apparatus at least to process EEG signals obtained from a subject using a grid of electrodes to determine a phase coherence measure of the EEG signals for selected electrode pairs of said grid; determine electrode pairs where strong phase coherence of the EEG signals is observed in a low frequency range during interictal activity; and use the determined electrode pairs to identify a possible seizure zone of the subject's brain.

As the seizure state is associated with enhanced neuronal synchronization [5], coherence techniques present an attractive approach for the study of coupling patterns in various frequencies in the epileptic brain. Many studies have investigated the coherence of slower rhythms (i.e. <80 Hz) during seizures [5], demonstrating increased coherence during the ictal state. As several studies have explored wavelet phase coherence (WPC <80 Hz), in the subject application WPC analysis is applied to both slow and fast neuronal rhythms (1 to 400 Hz) to study their respective temporal and spatial coherence patterns.

The subject method and apparatus provide advantages in that seizure zones can be identified using low frequency signal characteristics during non-seizure times. This is due to the fact that it has been found that strong coherence of low frequencies between certain electrode pairs coincide with increased coherence of high frequencies between the same electrode pairs. This allows seizure zones to be identified without the need for a seizure to occur. Also, as typical EEG equipment in many hospitals is only suitable for detecting low frequency brain activity, the subject method and apparatus can be employed by these hospitals using their existing equipment to identify seizure zones. Furthermore, the identification of seizure zones using low frequency characteristics can be used to supplement the identification of seizure zones using high frequency characteristics thereby to pinpoint smaller regions of brain tissue to be removed and avoid excess tissue removal and to reduce the need for secondary surgeries.

### BRIEF DESCRIPTION OF THE DRAWINGS

Embodiments will now be described more fully with reference to the accompanying drawings in which:

FIG. 1 shows (A) placement of a subdural electrode grid in patient 1 and the numbering of electrode contacts; (B) an iEEG recorded from all electrodes of the implanted electrode grid during one seizure episode (duration=240 seconds); (C) an iEEG recorded from electrode 10 of the implanted electrode grid during interictal activity, magnified at right; and (D) an iEEG recorded from electrode 10 during seizure activity, magnified at right with electrode 10 being indicated in gray on the implanted electrode grid;

FIG. 2 shows (A) an iEEG recorded from electrode 10 of the implanted electrode grid during interictal activity of patient 1; (B) a Wavelet phase coherence (WPC) profile for an electrode pairing comprising electrodes 9 and 10 (E9,E10) of the implanted electrode grid; (C1) a WPC from (B) magnified for the 1 to 30 Hz frequency range in which a strong WPC in the lower frequency range is visible for electrode pairing (E9, E10) during interictal activity; (C2) a WPC profile for electrode pairing (E23,E24) of the implanted electrode grid in which there is minimal coherence between electrode pairing (E23, E24) during interictal activity; (C3) an average WPC for the electrode pairings (E9, E10) and (E23, E24) over 120 seconds of interictal activity in which strong coherence is visible in the 8 to 11 Hz frequency band for electrode pairing (E9, E10) (black-dashed); and (D) an average WPC for two (2) second windows during interictal activity for the 8 to 11 Hz frequency band where averaged grids were computed for each electrode by averaging over all possible electrode pairings of the implanted electrode grid in the indicated time windows and frequency range and where averaged grids correspond spatially to the implanted electrode grid shown at right;

FIG. 3 shows (A) an iEEG recorded from electrode 10 of the implanted electrode grid during seizure activity of patient 1; (B) a Wavelet phase coherence (WPC) profile for electrode pairing (E9,E10) of the implanted electrode grid; (C) a frequency-normalized wavelet distribution of electrode 1 of the implanted electrode grid displaying increased spectral power during the seizure; (D1) a WPC from (B) magnified for the 120 to 130 second region in which strong WPC in the higher frequency range is visible for electrode pairing (E9, E10) during seizure activity; (D2) a WPC profile for electrode pairing (E23,E24) in which there is minimal coherence between electrode pairing (E9, E10) during seizure activity; (D3) an average WPC for the electrode pairings from D1 and D2 over the 10 seconds of seizure activity from D1, D2 where strong coherence is visible in the 100-250 Hz frequency band for electrode pairing (E9, E10) (black-dashed); and (E) an average WPC for one (1) second windows during seizure activity for the 100 to 250 Hz frequency band where averaged grids were computed for each electrode by averaging over all possible electrode pairings of the implanted electrode grid in the indicated time windows and frequency range and where averaged grids correspond spatially to the implanted grid shown at right;

FIG. 4 shows (A) strongly cohered electrode pairings (average WPC higher than the indicated threshold) in gray for each patient during low-frequency (LF) interictal and high-frequency (HF) seizure activity; (B) time-frequency averaged HF wavelet power matrices extracted from interictal and seizure segments for all electrodes on the implanted electrode grid where grid areas possessing the strongest HFO spectral power during seizures correspond to those possessing strong coherence;

FIG. 5 shows coherence profiles of interictal LFOs and ictal HFOs, with one electrode pairing, exhibiting elevated coherence during non-seizure and seizure activity, being shown for each patient and the average WPC (over the entire plotted time segment) being displayed to the right of each WPC plot;

FIG. 6 shows mean WPC (1 to 80 Hz) during interictal activity for one electrode pairing for each patient, the mean WPC being averaged in time for one interictal segment for the indicated electrode pairings at the right and with frequencies >30 Hz being expanded at the right;

FIG. 7 shows mean interictal LFO (5 to 12 Hz) WPC matrices, with the mean interictal LFO WPC values calculated for all possible electrode pairings (left column), with matrix values plotted as histograms (middle) to identify strongly cohered electrode pairings and with suprathrehold electrodes (i,e, electrodes involved in the pairings exhibiting coherence interactions greater than the indicated thresholds) highlighted on the grid at the right;

FIG. 8 shows spatial locations of cohered suprathreshold electrodes during seizure and non-seizure activity;

FIG. 9 shows LFO and HFO WPC values averaged over the indicated time windows and frequency bands during non-seizure and seizure activity for patient 1, with the strongest mean LFO coherence persisting in a given cluster of electrodes (top) and mean HFO coherence increasing and remaining highest in a similar area of the grid during seizure activity;

FIG. 10 shows mapping of clinically marked seizure onset zones (SOZs) of neurologists A and B with LFO/HFO defined regions of interest (ROIs); and

FIG. 11 shows mapping of hypothesized tissue resection areas with LFO/HFO defined ROIs with the resected electrodes corresponding to the SOZs identified by neurologist A.

### DETAILED DESCRIPTION OF EMBODIMENTS

In the following, a method, apparatus and computer-readable medium for processing EEG signals obtained from a subject using a grid or array (hereinafter referred to as grid) of electrodes is described. During the method, the EEG signals are processed to determine a phase coherence measure of the EEG signals for selected electrode pairs of the grid. Electrode pairs, where strong phase coherence of the EEG signals is observed in a low frequency range during interictal activity, are determined. The determined electrode pairs are used to identify a possible seizure zone of the subject's brain.

The EEG signals are processed using a suitable computing device. In this embodiment, the computing device is a general purpose computer or other suitable processing device comprising, for example, a processing unit comprising one or more processors, system memory (volatile and/or non-volatile memory), other non-removable or removable memory (e.g., a hard disk drive, RAM, ROM, EEPROM, CD-ROM, DVD, flash memory, optical data store etc.) and a system bus coupling the various computing device components to the processing unit, The memory of the computing device stores program instructions, that when executed, causes the computing device to process EEG signals obtained from a subject using a grid of electrodes as described above. A user may enter input or give commands to the computing device via a mouse, keyboard, touch-screen or other suitable input device. Other input techniques such as voice or gesture-based commands may also be employed to allow the user to interact with the computing device.

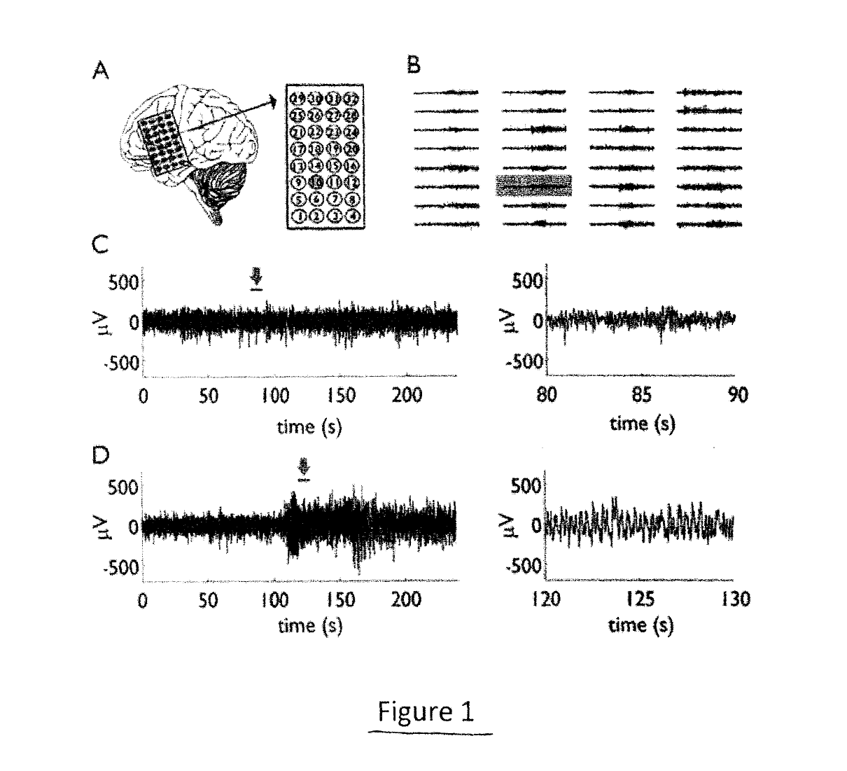

The subject method will now be described with reference to iEEG data acquired from a plurality of patients. In one study, iEEG data were collected from three (3) patients at the Thailand Comprehensive Epilepsy Program, Phramongkutklao Hospital (Bangkok, Thailand). All patients presented with extratemporal lobe epilepsy, and underwent presurgical evaluation for epilepsy surgery. Patients underwent surgery for the positioning of intracranial electrode grids, arranged in a sixty-four (64) contact (8×8) grid pattern (PMT, Chanhassen, Minn., U.S.A.) with the intracranial electrode grids being placed directly on the cortical surface. The iEEG recordings were performed with video monitoring and consisted of activity recorded during seizures and in between epileptic events. AH iEEG recordings were sampled at 2000 Hz (Stellate, Montreal, QC, Canada). The iEEG recordings were referenced to an electrode located at the forehead or behind the ears, but subsequently arranged offline in a bipolar arrangement in order to diminish artifacts. The bipolar arrangement consisted of taking the difference between pairs of neighboring electrodes, thereby reducing the number of electrodes for analysis to thirty-two (32) as shown in FIG. 1A. Electrical noise, 50 Hz and harmonics, was removed using finite impulse response (FIR) notch filtering. All analyses were performed by the computing device using MATLAB (The MathWorks, Natick, Mass., U.S.A.).

There is a general interest in studying synchronous activity by means of phase analyses. In contrast to classical coherence techniques, phase coherence allows for the separation of phase components from amplitude for a given frequency or frequency range. As faster brain activities are associated with lower amplitudes, phase coherence thus presents an effective tool for the study of all frequencies in general and higher frequency activities in particular.

Phase coherence involves the estimation of the instantaneous phases of electrical brain signals followed by a statistical method for quantifying the degree of phase locking. In this embodiment, the method for obtaining a phase coherence measure from the continuous wavelet transform follows from the work of Mormann et al. [6]. The original real-valued EEG signals are transformed into complex-valued signals by convolution with a complex wavelet [7]. Wavelet phase coherence (WPC) is performed for a chosen frequency value, around which a frequency range is defined. The process is repeated for all frequency values of interest until the entire portion of the spectrum under investigation has been covered. The phases of the signals are obtained from the coefficients of their wavelet transform at the frequency of interest. The coefficients result from the convolution of the raw signals with a scaled wavelet whose center frequency is in the center of the band of interest. At each time t and frequency f, the result of the convolution is a complex number A(t,f)e^{iφ}(t,f), where A is the amplitude and φ the phase of the signal. The phase difference of two signals (xa and xb) at a phase locking ratio of 1:1, for a given frequency is given by:

φ_{1,1}(t)=φₓₐ(t)−φ_{xb}(t)

Thus, the phase relationship between the wavelet coefficients of two different signals at a given scale corresponds to the phase relationship between the signals themselves at the frequency represented by that scale. Consequently, the relative phase coherence between two signals for a given frequency f or scale, time segment centered at time t=tₖ, and phase difference φ_{1,1} (at the phase locking ratio of 1:1), is obtained as follows:

$$ρ ( f , {t}_{k} ) = \vert 〈 exp ( {i∅}_{1 , 1} ( f , {t}_{k} ) ) 〉 \vert = \vert \frac{1}{( N + 1 )} \vert {\sum }_{j = k - N / 2}^{k + N / 2} exp ( {i∅}_{1 , 1} ( f , jΔ t ) )$$

The relative phase coherence varies between 0 (independent signals) and 1 (constant phase-lag between two signals). In this embodiment, WPC was calculated for frequencies between 1 to 400 Hz at a resolution of 1 Hz, using the complex Morlet wavelet A moving window of (1/f)*10 second duration was applied to each iEEG segment, where f is equal to the current frequency of interest. The window size was chosen large enough to contain several signal oscillations, yet brief enough to reduce smoothing. All possible electrode pairings were applied, resulting in a WPC matrix representing coherence as a frequency-time distribution for each electrode pairing.

MRI findings for the three patients (16-36 years, epilepsy duration from 4-20 years) from which the iEEG date were obtained are summarized in Table 1 below:

TABLE 1
PATIENT CHARACTERISTICS OF INTRACRANIAL EEG DATA

| | | Epilepsy | |
|---|---|---|---|
| | | Duration | |
| Patient | Age/Sex | (years) | MRI Findings |
| 1 | 36/F | 20 | Abnormal |
| | | | intensity |
| | | | lesion, |
| | | | perisylvanian |
| 2 | 16/M | 4 | Cortical |
| | | | dysplasia |
| 3 | 30/M | 16 | Hippocampalatrophy, |
| | | | dilated |
| | | | perisylvanian |

Ictal and interictal segments of iEEG data were analyzed from all patients. Seven seizure segments in total were analyzed (at least two seizure episodes were included for each patient). Each seizure segment contained a single seizure episode, as well as at least one (1) minute of data preceding and following the seizure (mean duration of each seizure segment: four (4) minutes). Interictal segments were obtained from data recorded in between seizure episodes. In total eight (8) minutes of interictal data was analyzed from patient 1, seven and one-half (7.5) minutes of interictal data was analyzed from patient 2 and seventeen (17) minutes of interictal data was analyzed from patient 3. The shortest interictal segment was one and one-half (1.5) minutes in the length and the longest interictal segment twelve (12) minutes. An example of an interictal and seizure recording from patient 1 is plotted in FIGS. 1C and 1D.

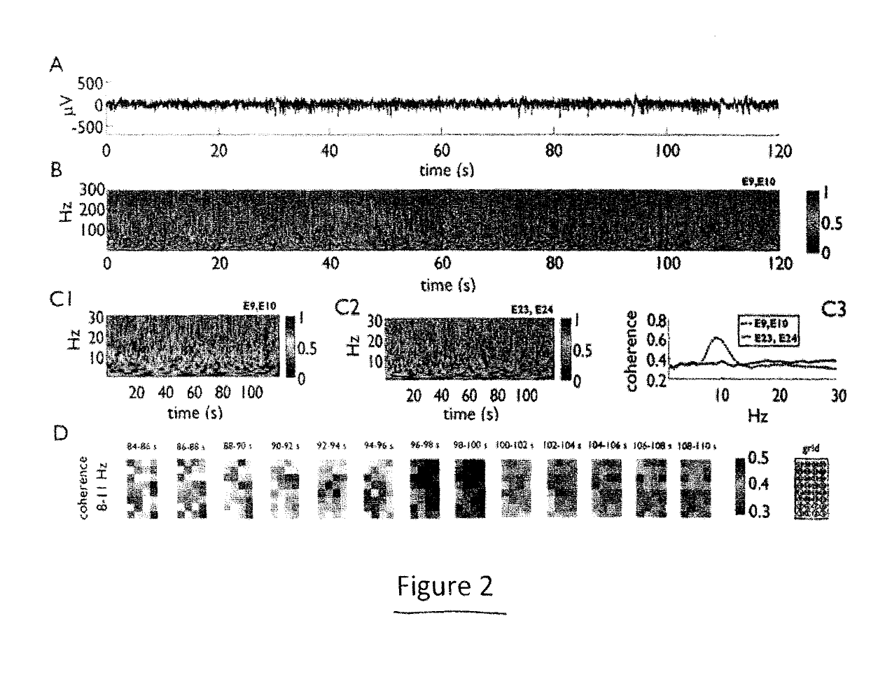

WPC between all possible electrode pairings was performed. In analyzing all of the WPC matrices, two prominent patterns of phase coherence were observed. During interictal activity, strong phase coherence was observed in the 8 to 11 Hz frequency range, while increased phase coherence was observed in activity >80 Hz during seizure episodes. FIGS. 2B, 2C1 and 2C2 depict the WPC profiles of two electrode pairings during interictal activity from patient 1. While coherence is visible in other frequency bands, the strongest coherence is visible in the slower rhythms. As will be appreciated, not all electrode pairings exhibit this pattern of activity. Neighboring electrodes 9 and 10 show marked coherence in the 8 to 11 Hz band over a two (2) minute span, while minimal coherence in the same frequency band is present in neighboring electrodes 23 and 24. In analyzing various interictal segments from all patients, it was observed that this coherence pattern of low-frequency activity appeared in selective electrodes. Furthermore, this activity appeared for longer time intervals in some patients, as depicted in FIG. 2, and for shorter time intervals in other patients.

Globally averaged WPC matrices were computed for interictal periods by averaging over time, frequency and electrode pairings. This provided a measure of global coherence for each electrode. Each box in the matrix of FIG. 2D corresponds spatially to one electrode from the implanted electrode grid. The average coherence of the electrode with all other electrodes is represented by the bar (see the right hand side of FIG. 2D). Matrices were averaged over two (2) second time windows and over the 8 to 11 Hz frequency band. Each global electrode value was computed by averaging over all possible electrode pairings for the electrode in the 2-second time windows and a frequency range of 8 to 11 Hz. The strongest coherence was observed in the lower left quadrant of the implanted electrode grid.

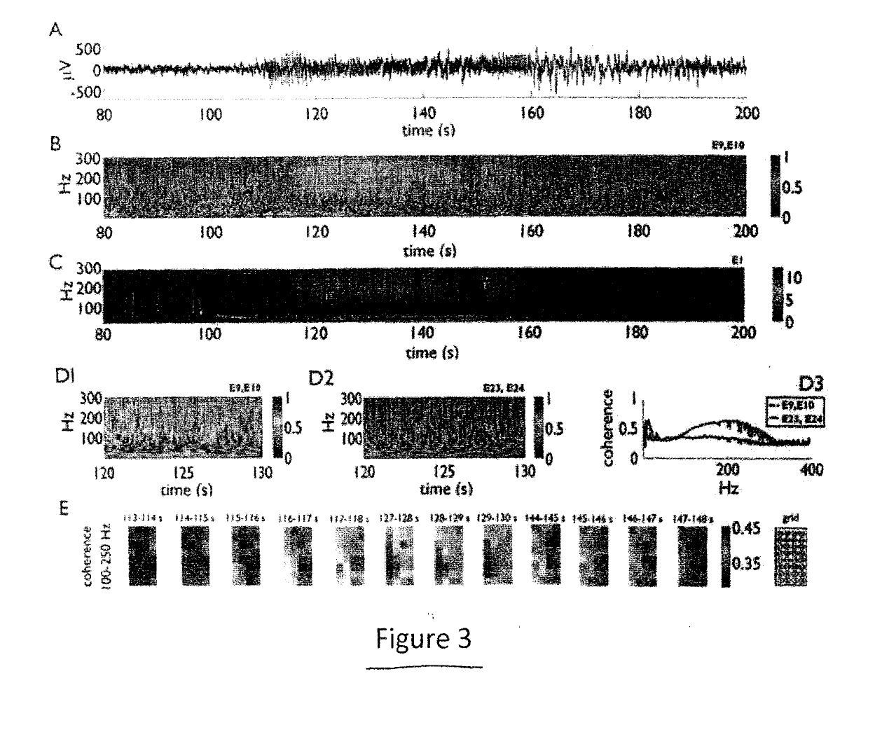

FIGS. 3B, 3D1 and 3D2 depict the WPC profiles of the two electrode pairings of FIG. 2 during seizure activity from patient 1. Once again, while coherence is visible in other frequency bands, strong coherence was visible in the faster rhythms (i.e. >80 Hz). As during interictal activity, not all electrode pairings exhibited this pattern of activity. Neighbouring electrodes 9 and 10 show marked coherence in the 100 to 250 Hz band during ictal activity, while minimal coherence in the same frequency band is present in neighbouring electrodes 23 and 24 (FIG. 3D3). Globally averaged WPC matrices were also computed for seizure episodes. Each global electrode value was computed by averaging over all possible pairings for the electrode in one (1) second time windows and a frequency range of 100 to 250 Hz. The strongest coherence was observed in the lower left quadrant of the implanted electrode grid during ictal activity.

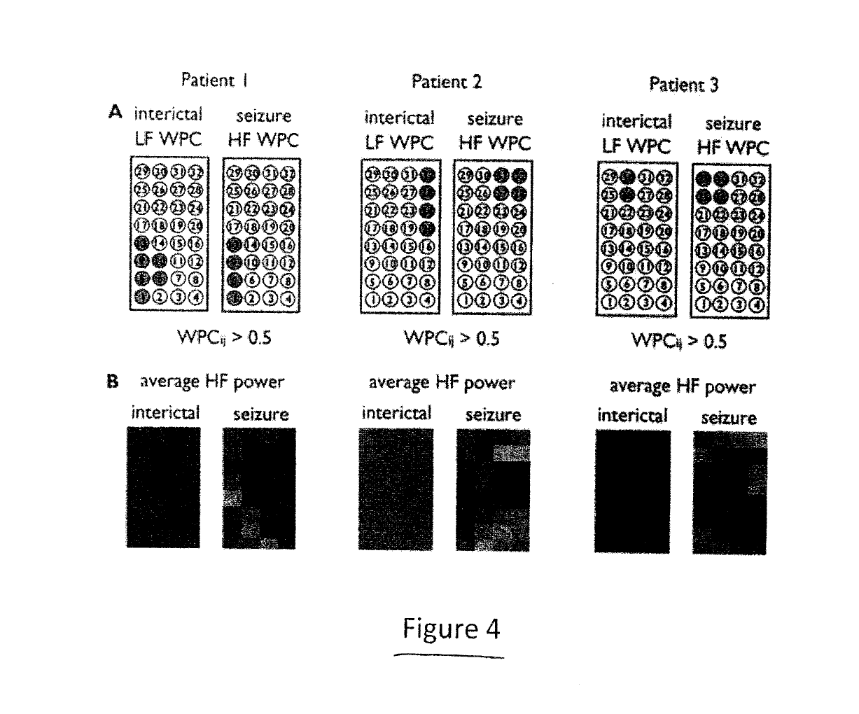

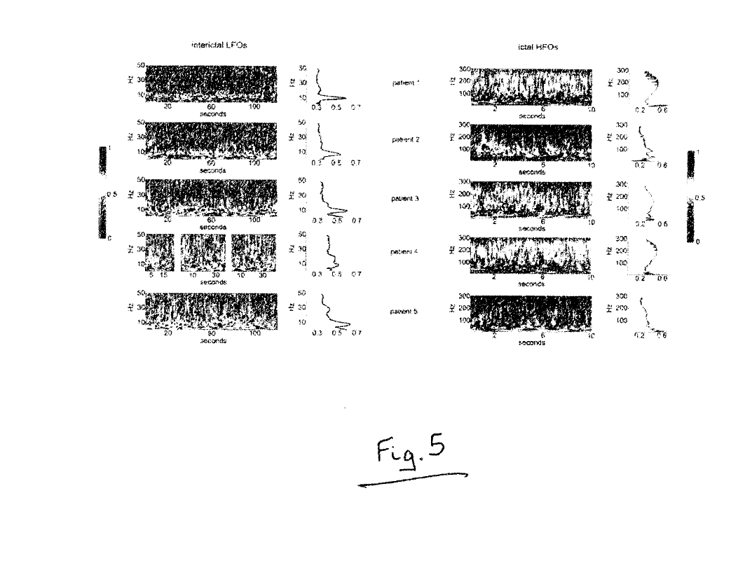

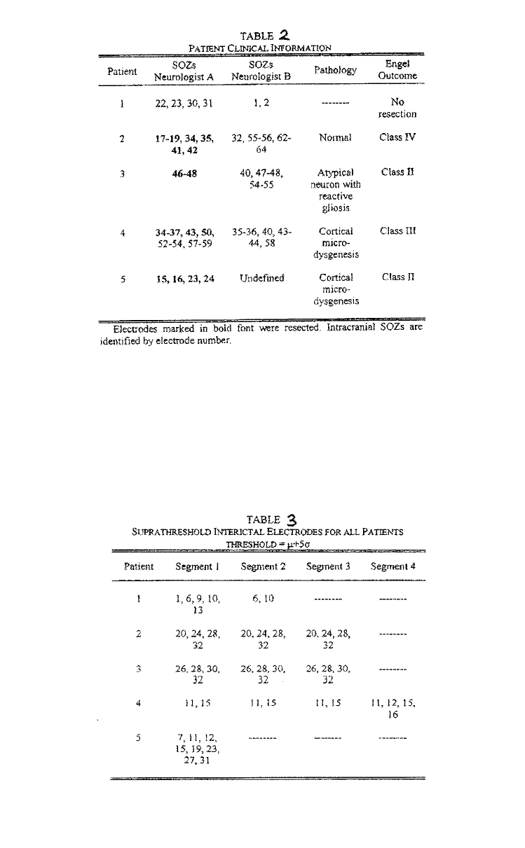

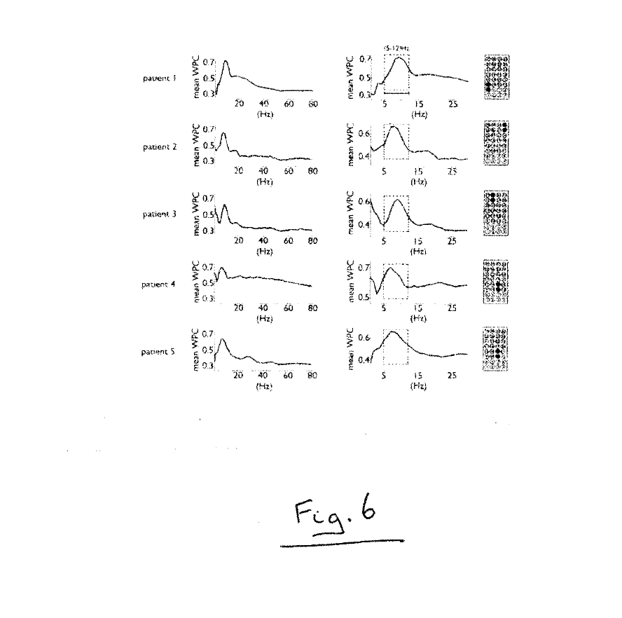

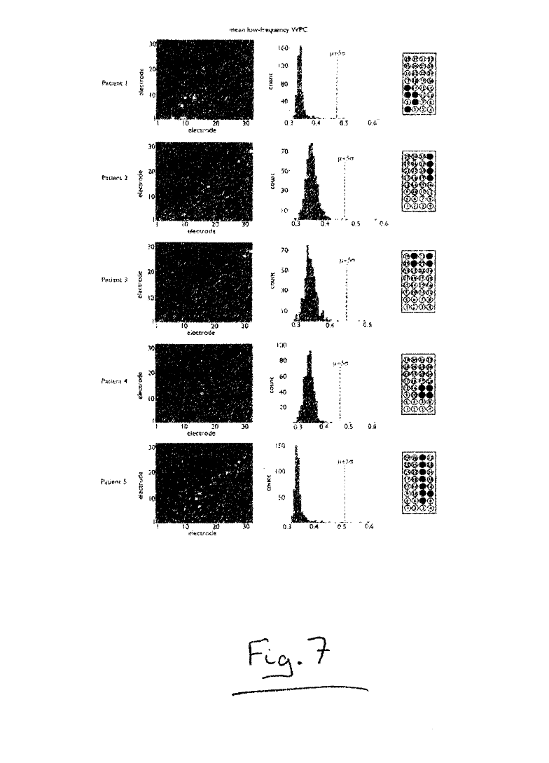

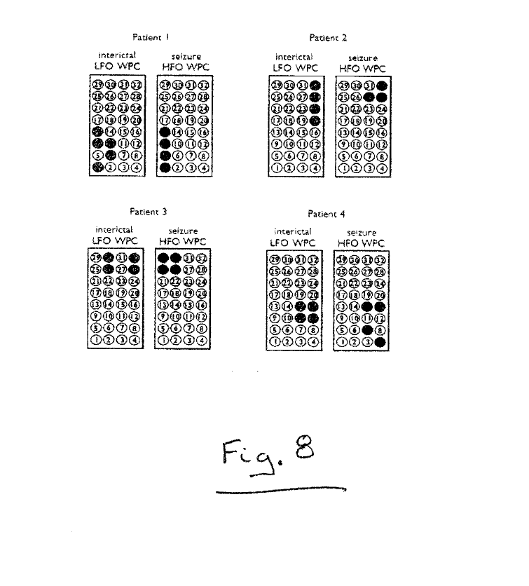

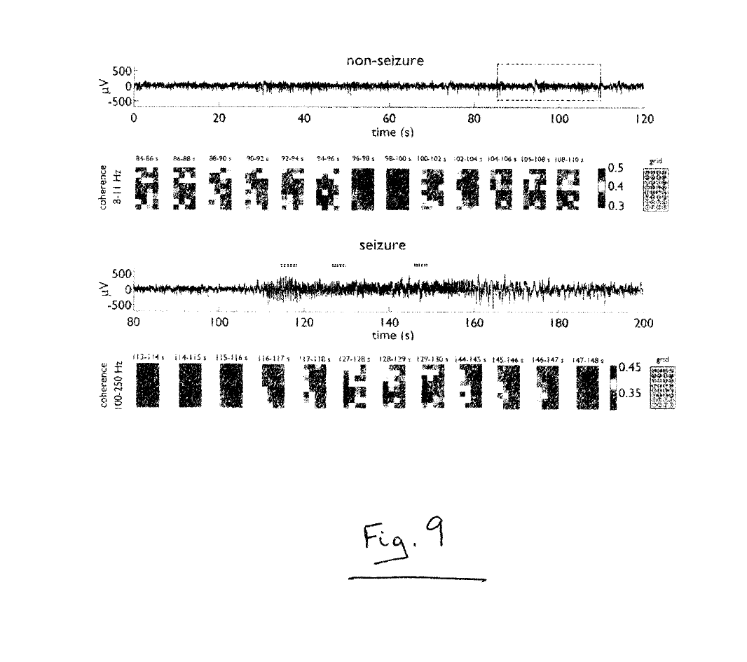

Strongly cohered electrode pairings (average WPC higher than the indicated threshold) were marked on each patient grid for interictal and seizure segments. Grid locations possessing strong HFO WPC during seizures showed overlap with areas possessing strong low-frequency coherence during interictal activity (FIG. 4). Frequency normalized HFO power was calculated in frequencies ranging from 80 to 400 Hz across patients (FIG. 3C), and similar to WPC, varied spatially across the implanted electrode grids. A select number of electrodes displayed the strongest rises in HFO power during seizures (FIG. 4A). Average spectral power matrices were computed for all three patients for interictal and ictal segments. In general, HFO power increases were observed in frequency bands similar to those showing increased coherence. Furthermore, areas on the electrode grids possessing the strongest HFO spectral power during seizures corresponded to those possessing elevated coherence (FIG. 4).

Osculatory processes are involved in the integration of local and distributed neuronal populations, and appear to abnormally dominate in epilepsy [8]. As will be appreciated, the WPC algorithm is a useful technique for the measurement of synchrony between brain rhythms. In this embodiment, WPC is applied to investigate the spatial and temporal patterns of neuronal coherence from iEEG data recorded in patients during interictal and seizure activity in the 1 to 400 Hz frequency range.

In each patient, patterns of strong phase coherence were observed. During interictal activity, strong coherence appeared in the 8 to 11 Hz range. During seizures, strong coherence appeared in activity >80 Hz. Furthermore, the above findings demonstrated that regions of focally enhanced neuronal coherence during interictal activity (8 to 11 Hz) overlapped with cohered ictal HFO activity (>80 Hz). Also, electrodes involved in pairings exhibiting strong HFO coherence during seizures, possessed increased HFO spectral power ictal changes as well.

Visually, large-amplitude rhythmic fluctuations (spike-and-wave discharges) are readily apparent in the iEEG of epilepsy patients, indicative of an underlying excitatory and co-ordinated network [5]. As such, numerous human studies have performed analyses on the coherence of neuronal rhythms (i.e. <80 Hz) during seizures, demonstrating a correlation between enhanced coherence and seizure activity [5, 6]. A smaller subset of studies have also demonstrated elevated synchrony in iEEG recordings of interictal activity, separate from epileptiform disturbances [3, 9]. In particular, similar to the results presented here, Le Van Quyen et al. [9] have shown that the synchrony of electrode pairings near the primary epileptogenic zone, in the 4 to 15 Hz frequency range, increased or decreased before seizures.

The involvement of HFOs during seizures is poorly understood. Their appearance in local field potentials recorded from humans and rodents has been associated with the epileptogenic zone and highlights them as an attractive target for the identification of the epileptogenic zone [4]. As such, several studies have shown a correlation between the removal of regions with ictal HFOs increases and a good post-surgical outcome [3]. This would suggest that HFO increases in amplitude, and analogously in spectral power, help to delineate the epileptogenic zone. The subject method demonstrates that ictal HFO spectral power increases spatially correlated with cohered HFO activity on the implanted patient grids.

The spatial correlation observed between regions of elevated coherence during interictal periods in slower rhythms with that of elevated HFO coherence during seizures suggests the ability of interictal recordings to contribute to the identification of the epileptogenic zone.

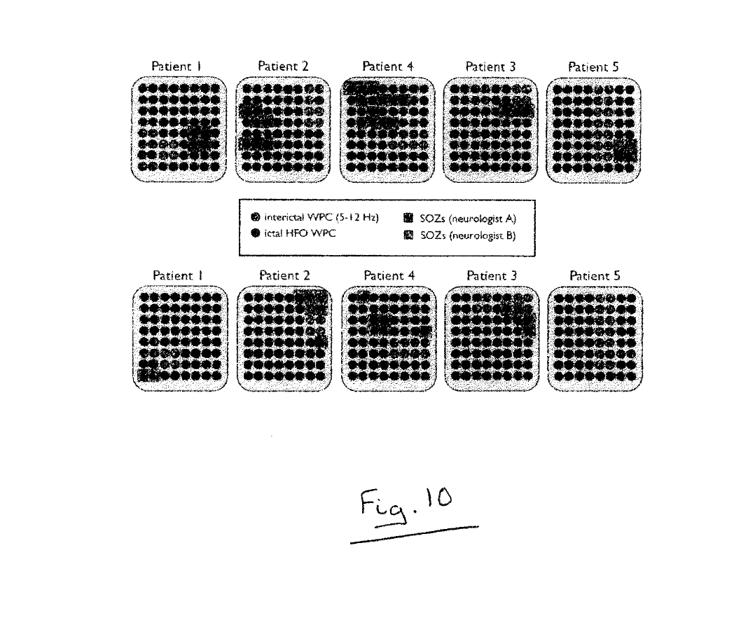

In a supplemental study, the iEEG data of five (5) patients were analyzed. The recorded iEEG data were independently reviewed off-line by two neurologists (neurologists A and B, Table 3, FIG. 10) to clinically delineate seizure onset zones (SOZs) for all five patients. SOZ identification (performed by both neurologists) was completed separately. The SOZs identified by both neurologists were defined electrographically as the electrode(s) with the earliest seizure activity. In addition, neurologist B was blinded to all clinical information available from the pre-surgical planning phase.

Four of the five patients studied undertook epilepsy surgery. Brain tissue resection was limited to the areas subjacent to electrodes located in the electrographically defined SOZs (according to the SOZs defined by neurologist A). Patient 1 did not undergo surgery due to the close proximity of the SOZ to eloquent cortex. Patient 4 underwent a limited resection as a portion of the SOZ was also in close proximity to eloquent cortex (see SOZs and resected regions, Table 2). Each parent's surgical outcome was categorized according to Engel's classification [10] as described in Table 2: a) class 1: free, b) class 2: rare disabling seizures, c) class 3: worthwhile improvement and d) class 4: no worthwhile improvement

Similarly, WPC was calculated for frequencies between 1-400 Hz at a resolution of 1 Hz, using the complex Morlet wavelet. A moving window of (1/f)*10 second duration was applied to each iEEG segment, where f is equal to the current frequency of interest. The window size was chosen large enough to contain several signal oscillations, yet brief enough to reduce smoothing. All possible electrode pairings were applied, resulting in a WPC matrix representing coherence, as a frequency-time distribution, for each electrode pairing.

Ictal and interictal segments were analyzed for all patients (n=5). Thirteen seizures in total were analyzed (at least two seizures were included for each patient). Each iEEG seizure segment consisted of a single seizure episode, and at least one (1) minute of data preceding and following the seizure (mean duration of each seizure segment: 264 seconds). Interictal segments were obtained from iEEG data recorded in between seizure episodes. A total of 33.5 minutes of interictal data was analyzed from all five patients.

Wavelet phase coherence was calculated for interictal and ictal iEEG segments, for all possible electrode combinations. The WPC profiles of HFO activity showed minimal variations over time and space during interictal activity, in all five patients. HFO (80-300 Hz) coherence was consistently transient and of weak to moderate strength during non-seizure activity, for all electrode pairs. In contrast, high HFO WPC values were observed in select electrode clusters, during seizures, for ⅘ patients. Ictal WPC profiles are shown in FIG. 5 for all five patients. Each plot represents one of the most strongly cohered electrode pairings from each patient during a seizure episode. Electrode pairings from patient 5 did not exhibit strongly cohered HFO pairings.

Strong LFO WPC values were observed during ictal and interictal activity in all five patients; however, in studying the non-seizure and seizure segments from all patients, it was observed that elevated LFO coherence did not show a spatial selectivity during seizures. High LFO WPC values were only observed in select electrode clusters, during interictal activity, for all five patients. Interictal WPC profiles are shown in FIG. 5 for all five patients. Each plot represents one of the most strongly cohered electrode pairings from each patient during non-seizure activity.

Electrodes possessing strong HFO ictal coherence were characterized and identified as described in [11]. Briefly, the HFO bandwidth was bounded at frequency values located at 0.37WPCₘₐₓ, where WPCₘₐₓ was the peak WPC value calculated for each electrode pairing. While frequency bandwidths and peak frequencies varied in space and time, and across seizures and patients, the defined HFO bandwidth for each patient was based upon the widest frequency range of HFO activity identified across all electrodes in the implanted grids and across all recorded time intervals. WPC values were averaged across frequency and time to generate a matrix consisting of average WPC strength for each electrode pairing. To isolate HFO activity, the averaging was completed using the defined HFO frequency bands for each patient (as described above). A comprehensive exploration of all electrode pairings on the implanted subdural grids, during seizure activity, yielded the spatial locations of strongly cohered electrode clusters, HFO regions of interest (ROIs), in ⅘ patients (FIG. 8).

Electrodes possessing strong LFO interictal coherence (i.e. electrode pairings with mean LFO-WPC values greater than the indicated thresholds in Table 3) were further explored to elucidate the frequency spread of cohered LFO activity. While the bandwidths of low-frequency activity varied in space and time, the defined LFO bandwidth for each patient were based upon the widest frequency range of LFO activity identified across all electrode clusters possessing strong LFO coherence. In FIG. 6, mean LFO bandwidths are plotted for all patients, for the indicated electrode pairings. WPC was averaged in time, over the entire duration of the plotted interictal activity. A low-frequency bandwidth of 5-12 Hz was chosen to capture LFO-WPC changes across all patients.

A comprehensive study of all electrode pairings on the implanted subdural grids, during interictal activity, yielded the spatial locations of strongly cohered (LFO) electrode clusters for all patients. WPC values were averaged across frequency and time to generate a matrix consisting of an average WPC estimate, for each electrode pairing. To isolate LEO activity, the averaging was completed using only the low-frequency bands (5-12 Hz) of each patient. The matrices, for one interictal segment from each patient, are shown in FIG. 7 (left). (Note, as each matrix is symmetric, only half of each matrix is displayed for clarity). The mean seizure LFO WPC values (from the matrices at left) are also plotted as histograms (FIG. 7, middle). Electrode pairings with LEO coherence values greater than the indicated thresholds are highlighted at right (black circles). For all interictal segments and patients, a threshold of t+5σ (where t is the mean and o the standard deviation of the mean LFO-WPC values for each interictal segment) highlighted clusters of electrodes with strongly cohered low-frequency (5-12 Hz) activity. The suprathreshold electrodes (for thresholds=t+5σ) for all interictal segments and all patients are listed in Table 3.

Strongly cohered HFO and LFO electrode pairings (i.e. suprathreshold electrodes, where average WPC was higher than the indicated thresholds) are marked on the patient grids in FIG. 8. Electrode clusters possessing strong HFO WPC during seizures and strong LFO WPC during interictal activity highlight similar electrodes on the patient grids.

Average HFO and LFO WPC was computed to qualitatively characterize the spatiotemporal coherence patterns of HFO/LFO activity. WPC values, in the indicated LFO and HFO frequency bands were averaged in space (across all possible electrode pairs), and in time (within 1 and 2 second windows), yielding a global WPC mean value for each electrode. These average WPC values were arranged in the same spatial layout as the subdural grid electrodes. Average WPC values for electrode contacts from patient 1 are shown in FIG. 9. Consecutive time windows of spatially averaged HFO/LFO coherence are shown for various segments of the plotted iEEG activity (i.e. seizure and non-seizure activity). While it was observed that the mean LFO coherence varied in time and space, the strongest mean LFO coherence persisted in a given cluster of electrodes. Furthermore, HFO coherence increased and remained highest in a similar area of the patient grid during seizure activity.

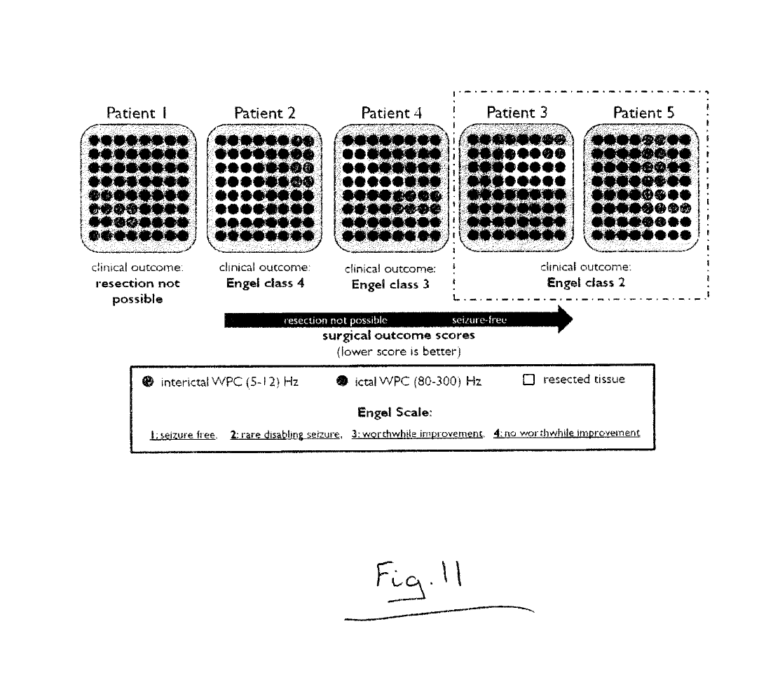

The spatial location of electrodes exhibiting strong LFO interictal coherence and strong ictal HFO coherence (from FIG. 8) are mapped as HFO/LFO defined ROIs in FIGS. 10 and 11. The LFO and HFO defined ROIs are mapped onto the clinical SOZ electrodes identified by neurologists A and B (Table 2) in FIG. 10 and over the surgically excised electrodes in FIG. 11. The resected electrodes corresponded to the SOZs identified by neurologist A and covered an additional 1 to 2 electrode contacts unless the SOZ was found to be in proximity to functional/eloquent cortex.

In general, the SOZs defined by both neurologists did not always match. Neurologists A and B marked similar SOZs for patients 3 and 4, yet their SOZs noticeably differed for patients 1 and 2. Furthermore, neurologist B determined that the brain region responsible for seizure onset was not discernible in the iEEG of patient 5, and concluded that the SOZ originated in an area not covered by the implanted grid. As a result, no SOZs are defined for patient 5 (in FIG. 10) for neurologist B. It was observed that the LFO/HFO defined ROIs were typically in close proximity or overlapping with the clinically marked SOZs of at least one neurologist. In FIG. 11, post-surgical patient outcomes were ordered from worst to best, in terms of surgical scores (left to right) and ranged from the left side with patient 2 who possessed an Engel class 4 outcome (no worthwhile improvement) to the right end with patients 3 and 5 who both had Engel class 2 outcomes (rare disabling seizures). The best clinical outcome was observed when the patient resection areas and HFO/LFO-defined ROIs were in close proximity (i.e. patients 3 and 5). Similarly, a poorer surgical outcome was obtained when tissue resection areas and ROIs were incongruent (i.e. patient 2).

In this study a wavelet phase coherence analysis was applied to investigate whether electrode clusters demonstrating strong LFO coherence, relative to all other electrodes on the implanted patient grids, resided in tissue areas which exhibited increased ictal HFO coherence. Clusters of electrodes possessing strongly cohered LFOs during interictal activity were observed. In comparing these more active interictal LFO associated regions of the cortex with the ictal HFO ROIs defined in [12], it was observed that electrodes on the patient grids possessing elevated HFO coherence were similar to those possessing elevated LFO coherence during interictal activity (FIG. 8). Furthermore the best clinical outcome was observed when the patient resection areas and HFO/LFO defined ROIs were in close proximity (FIG. 11).

Traditionally, oscillations in the 10 Hz frequency range were considered an idling rhythm, as they decreased with movement or cognitive changes [13]. However, recent studies have suggested that alpha rhythms (8-12 Hz) play an important role in controlling cortical excitability by exerting an inhibitory effect on cortical processing [14]. In the epileptic brain, increases in the (4-9 Hz) frequency band have been reported in the EEG of epilepsy patients, relative to normal controls [15]. Furthermore, Le Van Quyen et al. [9] concluded that the synchrony of electrode pairings near the primary epileptogenic zone, in the (4-15) Hz frequency range, increased and decreased before seizure onset. Along the same lines, the presence of high theta activity (i.e. 4-8 Hz) in awake adults is suggestive of abnormal and/or pathological activity [16]. As HFOs have been shown to highly localize to SOZs [17, 18], the overlapping spatial regions, which exhibited both increased coherence in ictal HFOs and interictal LFOs in the (5-12 Hz) frequency band, may identify local abnormalities that underlie epileptogenic networks.

The HFO and LFO guided coherence techniques described above provide potential epilepsy biomarkers, to complement those already available in the literature and can potentially be used to support the pre-surgical planning phase, when determining SOZs.

Although use of an 8×8 electrode grid is described above to obtain EEG data from patients, those of skill in the art will appreciate that alternative electrode grid arrangements may be employed. EEG data may be processed by the computing device off-line after the EEG data has been acquired and stored to memory or may be processed on-line as the EEG data is being acquired.

Although above embodiments employ the complex Morlet wavelet, those of skill in the art will appreciate that other wavelets, for example, complex Gaussian, frequency b-spline, complex Shannon etc. may be employed.

Although embodiments have been described with reference to the accompanying drawings, those of skill in the art will appreciate that variations and modifications may be made without departing from the scope thereof as defined by the appended claims.

### REFERENCES

[1] X. J. Wang, “Neurophysiological computational principles of cortical rhythms in cognition,” Physiological reviews, vol. 90, pp. 1195-268, July 2010.
[2] J. R. Hughes, “Gamma, fast and ultrafast waves of the brain: their relationships with epilepsy and behaviors,” Epilepsy and behaviour: E& B, vol. 13, pp 25-31, July 2008.
[3] J. Jacobs, R. Staba, E. Asano, H. Otsubo, J. Y. Wu, M. Zijlmans, et al., “High-frequency oscillations (HFOs) in clinical epilepsy,” Prog Neurobiol, Apr. 3, 2012.
[4] J. G. Jefferys, L. Menendez de la Pride, F. Wendling, A. Bragin, M. Avoli, I. Timofeev, et al., “Mechanisms of physiological and epileptic FIFO generation,” Prog Neurobiol, vol. 98, pp. 250-64, September 2012.
[5] P. J. Uhlhaas and W. Singer, “Neural synchrony in brain disorders: relevance for cognitive dysfunctions and pathophysiology,” Neuron, vol. 52, pp. 155-68, Oct. 5, 2006.
[6] F. Mormann, K. Lehnertz, P. David, and C. E. Elger, “Mean phase coherence as a measure for phase synchronization and its application to the EEG of epilepsy patients,” Physica D., vol. 144, pp. 358-369, 2000.
[7] O. C. Zalay, “Cognitive Rhythm Generators for Modeling and Modulation of Neuronal Electrical Activity ” PhD Doctoral Dissertation, Institute of Biomaterials and Biomedical Engineering, University of Toronto, 2012.
[8] G. Buzsaki and A. Draguhn, “Neuronal oscillations in cortical networks,” Science, vol. 304, pp. 1926-9, Jun. 25, 2004.
[9] M. Le Van Quyen, J. Soss, V. Navarro, R. Robertson, M. Chavez, M. Baulac, et al., “Preictal state identification by synchronization changes in long-term intracranial EEG recordings,” Clin Neurophysiol, vol. 116, pp. 559-68, March 2005.
[10] J. Engel and T. Rasmussen, “Outcome with respect to seizures,” in Surgical treatment of the epilepsies, J. Engel, Ed., 2nd ed New York: Raven Press, 1993.
[11] M. Cotic, O. Zalay, Y. Chinvarun, M. del Campo, P. L. Carlen, and B. L. Bardakjian, “Spatiotemporal Profiles of Electrical Rhythmic Activity in Human Extratemporal Lobe Epilepsy,” PhD Doctoral Dissertation, Institute of Biomaterials and Biomedical Engineering, University of Toronto, 2014.
[12] M. Cotic, O. Zalay, P. L. Carlen, Y. Chinvarun, and B. L. Bardakjian “Synchrony of high frequency oscillations in the human epileptic brain,” Conf Proc IEEE Eng Med Biol Soc, vol. 2013, pp. 5582-5, 2013.
[13] R. Hebert, a Lehmann, G. Tan, F. Travis, and A. Arenander, “Enhanced EEG alpha time-domain phase synchrony during Transcendental Meditation: Implications for cortical integration theory,” Signal Processing, vol. 85, pp. 2213-2232.
[14] S. Sadaghiani, R. Scheeringa, K. Lehongre, B. Morillon, A. L. Giraud, M. D'Esposito, et al., “alpha-band phase synchrony is related to activity in the fronto-parietal adaptive control network,” J Neurosci, vol. 32, pp. 14305-10, Oct. 10, 2012.
[15] L. Zhao, W. Wu, Z. Liang, and G. Hu, “Changes in EEG measurements in intractable epilepsy patients with neurofeedback training,” Progress in Natural Science, vol. 19, pp. 1509-1514, Nov. 10, 2009.
[16] H. Adeli, Z. Zhou, and N. Dadmehr, “Analysis of EEG records in an epileptic patient using wavelet transform,” J Neurosci Methods, vol. 123, pp. 69-87, Feb. 15, 2003.
[17] B. Crepon, V. Navarro, D. Hasboun, S. Clemenceau, J. Martinerie, M. Baulac, et al., “Mapping interictal oscillations greater than 200 Hz recorded with intracranial macroelectrodes in human epilepsy,” Brain: a journal of neurology, vol. 133, pp. 33-45, January 2010.
[18] J. Jacobs, P. LeVan, R. Chander, J. Hall, F. Dubeau, and J. Gotman, “Interictal high-frequency oscillations (80-500 Hz) are an indicator of seizure onset areas independent of spikes in the human epileptic brain,” Epilepsia, vol. 49, pp. 1893-907, November 2008.

1. A method of processing EEG signals obtained from a subject using a grid of electrodes comprising:
processing the EEG signals to determine a phase coherence measure of the EEG signals for selected electrode pairs of said grid;
determining electrode pairs where strong phase coherence of the EEG signals is observed in a low frequency range during interictal activity; and
using the determined electrode pairs to identify a possible seizure zone of the subject's brain.

2. The method of claim 1 wherein said selected electrode pairs comprise all possible electrode pairs of said grid.

3. The method of claim 1 wherein during said processing, wavelet phase coherence of the EEG signals for the selected electrode pairs is calculated.

4. The method of claim 3 wherein wavelet phase coherence is calculated for frequencies between 1 Hz and 400 Hz at a resolution of about 1 Hz.

5. The method of claim 4 wherein said wavelet phase coherence is calculated using a Morlet wavelet.

6. The method of claim 1 wherein the low frequency range comprises 8 Hz to 11 Hz.

7. The method of claim 1 further comprising, for the determined electrode pairs, determining if an increased phase coherence is observed in a high frequency range during seizure activity to verify the identification of the possible seizure zone.

8. The method of claim 7 wherein said high frequency range comprises frequencies greater than 80 Hz.

9. A non-transitory computer-readable medium comprising program code for, when executed, processing EEG signals obtained from a subject using a grid of electrodes comprising:
program code for processing the EEG signals to determine a phase coherence measure of the EEG signals for selected electrode pairs of said grid;
program code for determining electrode pairs where strong phase coherence of the EEG signals is observed in a low frequency range during interictal activity; and
program code for using the determined electrode pairs to identify a possible seizure zone of the subject's brain.

10. The computer-readable medium of claim 9 wherein said program code for processing determines wavelet phase coherence of the EEG signals for the selected electrode pairs.

11. The computer-readable medium of claim 10 wherein wavelet phase coherence is calculated for frequencies between 1 Hz and 400 Hz at a resolution of about 1 Hz.

12. The computer-readable medium of claim 11 wherein said wavelet phase coherence is calculated using a Morlet wavelet.

13. The computer-readable medium of claim 9 wherein the low frequency range comprises 8 Hz to 11 Hz.

14. The computer-readable medium of claim 8 further comprising, for the determined electrode pairs, program code for determining if an increased phase coherence is observed in a high frequency range during seizure activity to verify the identification of the possible seizure zone.

15. The computer-readable medium of claim 14 wherein said high frequency range comprises frequencies greater than 80 Hz.

16. An apparatus comprising:
memory storing executable instructions; and
at least one processor communicating with the memory and executing the instructions therein to cause the apparatus at least to:
process EEG signals obtained from a subject using a grid of electrodes to determine a phase coherence measure of the EEG signals for selected electrode pairs of said grid;
determine electrode pairs where strong phase coherence of the EEG signals is observed in a low frequency range during interictal activity; and
use the determined electrode pairs to identify a possible seizure zone of the subject's brain.

17. The apparatus of claim 16 wherein said selected electrode pairs comprise all possible electrode pairs of said grid.

18. The apparatus of claim 16 wherein during said processing, wavelet phase coherence of the EEG signals for the selected electrode pairs is calculated.

19. The apparatus of claim 18 wherein wavelet phase coherence is calculated for frequencies between 1 Hz and 400 Hz at a resolution of about 1 Hz.

20. The apparatus of claim 19 wherein said wavelet phase coherence is calculated using a Morlet wavelet.

21. The apparatus of claim 16 wherein the low frequency range comprises 8 Hz to 11 Hz.

22. The apparatus of claim 16 wherein said apparatus is further caused to, for the determined electrode pairs, determine if an increased phase coherence is observed in a high frequency range during seizure activity to verify identification of the possible seizure zone.

23. The method of claim 22 wherein said high frequency range comprises frequencies greater than 80 Hz.
